(19) United States
(12) Patent Application Publication
(10) Pub. No.: US 2022/0178918 A1
(43) Pub. Date: Jun. 9, 2022

(54) DEVICE FOR DETECTING SUBSTANCE TO BE MEASURED, AND METHOD FOR DETECTING SUBSTANCE TO BE MEASURED

(71) Applicant: CITIZEN WATCH CO., LTD., Tokyo (JP)

(72) Inventors: Kana WADA, Saitama (JP); Takaaki NOZAKI, Saitama (JP)

(21) Appl. No.: 17/440,767

(22) PCT Filed: Mar. 17, 2020
(86) PCT No.: PCT/JP2020/011825

§ 371 Date: Sep. 18, 2021

(30) Foreign Application Priority Data
Mar. 20, 2019 (JP) 2019-053626

(51) Int. Cl. G01N 33/543; G01N 33/569; G01N 21/64

(52) U.S. Cl. CPC G01N 33/54326; G01N 33/56961; G01N 2021/6439; G01N 21/6428; G01N 33/56916

(57) ABSTRACT

An object of a device and a method for detecting a substance to be measured according to an embodiment of the present disclosure is to conveniently detect a biological substance, such as a bacterium or a fungus. The detection device according to an embodiment of the present disclosure includes a container that retains a solution containing a substance to be measured and a magnetic labeling substance that binds specifically to the substance to be measured, a flow generating unit that generates a flow in a first direction at least in the solution, a magnetic field generating unit that generates a magnetic field gradient in the solution, and a detection unit that detects composite particles, based on motion of particles in a predetermined region in the solution, the composite particles including the substance to be measured and the magnetic labeling substance bound together.

### FIELD

The present invention relates to a device and a method for detecting a substance to be measured.

### BACKGROUND

There have been increasing needs for a method for detecting a biological substance, such as a virus, a bacterium, or a fungus, that exists in a solution of a biological sample. As a method for detecting a biological substance having a size of several hundreds of nanometers, such as a virus, is known an optical detection method with near-field light (e.g., Patent Literature 1). “Near-field light” refers to light generated, when light entering a low-refractive-index medium from a high-refractive-index medium is totally reflected by the interface, only near to the interface on the side of the low-refractive-index medium, and has the property of being rapidly attenuated as it goes away from the interface.

However it may be difficult to detect bacteria, fungi, or other biological substances by the optical detection method with near-field light because they have a size of several micrometers.

### CITATION LIST

### Patent Literature

Patent Literature 1: International Publication No. 2017/187744

### SUMMARY

An object of a device and a method for detecting a substance to be measured according to an embodiment of the present disclosure is to conveniently detect a biological substance, such as a bacterium or a fungus.

A device for detecting a substance to be measured according to an embodiment of the present disclosure includes a container that retains a solution containing the substance to be measured and a magnetic labeling substance that binds specifically to the substance to be measured, a flow generating unit (flow generator) that generates a flow in a first direction at least in the solution, a magnetic field generating unit (magnetic field generator) that generates a magnetic field gradient in the solution, and a detection unit (detector) that detects composite particles, based on motion of particles in a predetermined region in the solution, the composite particles being the substance to be measured to which the magnetic labeling substance is bound.

The predetermined region in the solution is preferably separated from an inner wall surface of the container.

The flow generating unit is preferably a light source that radiates spatial light into the container.

The solution may contain another substance that is not the substance to be measured nor the magnetic labeling substance, and the detection unit may detect the composite particles, based on motion of the composite particles and the other substance in the predetermined region in the solution.

The magnetic field generating unit preferably moves the composite particles in a second direction different from the first direction.

The magnetic field generating unit may move the composite particles in a second direction identical to the first direction.

The detection unit preferably detects the composite particles, based on directions of motion of the composite particles and the other substance.

The detection unit preferably detects the composite particles, based on speeds of motion of the composite particles and the other substance.

The flow generating unit may heat the solution to cause convection therein to generate the flow in the first direction at least in part of the solution.

The flow generating unit may rotate the container to generate the flow in the first direction at least in part of the solution.

The flow generating unit may stir the solution to generate the flow in the first direction at least in part of the solution.

The composite particles preferably further include a fluorescent labeling substance, and the detection unit preferably optically detects the fluorescent labeling substance to detect particles to which the fluorescent labeling substance is bound, and detects the composite particles, based on motion of the detected particles.

A method for detecting a substance to be measured according to an embodiment of the present disclosure includes the steps of retaining in a container a solution containing the substance to be measured and a magnetic labeling substance that binds specifically to the substance to be measured, generating a flow in a first direction at least in the solution, generating a magnetic field gradient in the solution, and detecting composite particles, based on motion of particles in a predetermined region in the solution, the composite particles being the substance to be measured to which the magnetic labeling substance is bound.

The detection device and method according to an embodiment of the present disclosure enable conveniently detecting a biological substance, such as a bacterium or a fungus.

### BRIEF DESCRIPTION OF DRAWINGS

FIG. 1 shows the configuration of a device for detecting a substance to be measured according to embodiment 1 of the present disclosure.

FIG. 2 shows the moving directions of the substance to be measured and other substances in a detection region in a solution detected by the detection device according to embodiment 1 of the present disclosure.

FIG. 3 is a flowchart for explaining the steps of a method for detecting a substance to be measured according to embodiment 1 of the present disclosure.

FIG. 4(a) is a side view of the detection device according to embodiment 1 of the present disclosure for explaining a trajectory of the substance to be measured in the detection device, and (b) is a top view of the detection region viewed from the side of a detection unit in (a).

FIGS. 5(a) to (c) are plan views of images obtained at different focal depths by the detection device shown in FIGS. 4(a), and (d) to (f) are side views of a container of the detection device corresponding to (a) to (c), respectively.

FIG. 6(a) is an image of the detection region in the solution captured by an imaging unit of the detection unit constituting the detection device according to embodiment 1 of the present disclosure, and (b) shows the luminance of detection light of the particles in the image of (a) obtained by image processing of a processing unit of the detection unit.

FIG. 7(a) is an initial image of the detection region in the solution captured by the imaging unit of the detection unit constituting the detection device according to embodiment 1 of the present disclosure, and (b) shows superposition of the initial image and an image obtained after the elapse of a predetermined time period from the capture of the initial image.

FIG. 8 shows the configuration of a device for detecting a substance to be measured according to modified example 1 of embodiment 1 of the present disclosure.

FIG. 9 shows the configuration of a stirrable container used in a device for detecting a substance to be measured according to modified example 2 of embodiment 1 of the present disclosure; (a) is a plan view, (b) is a side view, (c) shows how the container rotates at stirring, and (d) shows how the container rotates at detection of the substance to be measured.

FIG. 10(a) shows, with arrows, the positions and motion of particles at a certain time for the case that the container is rotated, and (b) shows the positions and motion of the particles after the rotation process with arrows.

FIG. 11 shows the configuration of a device for detecting a substance to be measured according to modified example 3 of embodiment 1 of the present disclosure.

FIG. 12 shows the moving directions of the substance to be measured and other substances in the detection region in the solution detected by the detection device according to modified example 3 of embodiment 1 of the present disclosure.

FIG. 13 shows the configuration of a device for detecting a substance to be measured according to embodiment 2 of the present disclosure.

FIG. 14 is a flowchart for explaining the steps of a method for detecting a substance to be measured according to embodiment 2 of the present disclosure.

FIG. 15(a) is a side view of the detection device according to embodiment 2 of the present disclosure for explaining a trajectory of the substance to be measured in the detection device, and (b) is a top view of a detection region viewed from the side of a detection unit in (a).

FIG. 16 shows the moving directions of the substance to be measured and other substances in the detection region in a solution detected by the detection device according to embodiment 2 of the present disclosure.

FIG. 17(a) is an image of the detection region in the solution obtained by the detection device according to embodiment 2 of the present disclosure, and (b) shows the luminance of detection light of the particles in the image of (a).

FIG. 18(a) is an initial image of the detection region in the solution captured by an imaging unit of the detection unit constituting the detection device according to embodiment 2 of the present disclosure, and (b) shows an image obtained after the elapse of a predetermined time period from the capture of the initial image.

FIG. 19(a) shows movement vectors with their initial points disposed at the origin of XY coordinates, and (b) shows movement vectors for the case that force in a first direction is zero with their initial points disposed at the origin of XY coordinate.

FIGS. 20(a) to (d) show the steps of measurement for the case that a fluorescent labeling substance is used in the detection method according to embodiment 2 of the present disclosure.

FIGS. 21(a) to (e) show the steps of measurement for the case that fluorescent staining is performed in the detection method according to embodiment 2 of the present disclosure.

FIG. 22(a) is a perspective view of a device for detecting a substance to be measured according to embodiment 3 of the present disclosure, and (b) shows an example of a display screen of a portable device for the case that the portable device is used as a detection unit.

FIG. 23 is a perspective view of the detection device according to embodiment 3 of the present disclosure in which a measurement housing is open.

FIG. 24 is a side view of examples of the container used in the detection devices according to embodiments 1 to 3 of the present disclosure; (a), (b), and (c) show side views of flat-bottomed, round-bottomed, and tapered containers, respectively.

FIG. 25 is a side view of the container and a magnetic field generating unit used in the detection devices according to embodiments 1 to 3 of the present disclosure; (a) shows an example in which they include a sharp-pointed magnetic field generating unit, and (b) shows an example in which the container includes a yoke in the case of (a).

### DESCRIPTION OF EMBODIMENTS

Hereinafter, devices and methods for detecting a substance to be measured according to embodiments of the present disclosure will be described with reference to the drawings. However, note that the technical scope of the present invention is not limited to these embodiments and includes the invention described in the claims and equivalents thereof.

### Embodiment 1

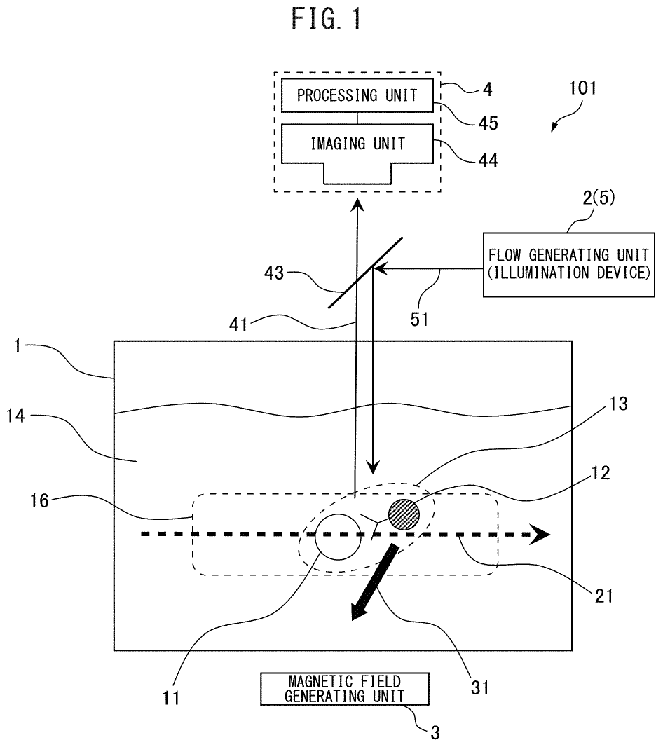

First, a device for detecting a substance to be measured according to embodiment 1 of the present disclosure will be described. FIG. 1 shows the configuration of a device 101 for detecting a substance to be measured according to embodiment 1 of the present disclosure. The detection device 101 according to embodiment 1 includes a container 1, a flow generating unit (flow generator) 2, a magnetic field generating unit (magnetic field generator) 3, and a detection unit (detector) 4.

The container 1 retains a solution 14 containing a substance 11 to be measured and a magnetic labeling substance 12 that binds specifically to the substance 11 to be measured. The magnetic labeling substance 12 preferably binds to all the substance 11 to be measured in the solution 14 to form composite particles 13. It is not necessary that these substances bind together at the very moment when the substance 11 to be measured and the magnetic labeling substance 12 are injected into the container 1. More specifically, for example, a flow of the solution 14 generated in the container 1 may facilitate a reaction by which the magnetic labeling substance 12 binds to the substance 11 to be measured, thereby generating the composite particles 13. Examples of the substance 11 to be measured include candida, Escherichia coli (E. coli), and CRP (C-reactive protein). Specific examples of the steps for detecting such a substance will be described below.

The flow generating unit 2 generates a flow in a first direction 21 at least in the solution 14. For example, as shown in FIG. 1, the flow generating unit 2 preferably generates a flow in the first direction 21 in a predetermined detection region 16 for detecting the composite particles 13 (also referred to simply as a “predetermined region”) in the solution 14. The predetermined region 16 is preferably separated from the inner wall surface of the container 1. Since the composite particles 13 are detected focusing on the region separated from the inner wall surface of the container 1, the composite particles 13 are not hindered from moving by their contact with the inner wall surface of the container 1. Further, the accuracy of detection can be improved by excluding composite particles adhering to the inner wall surface from the target of the detection process. Additionally, since the composite particles are detected focusing on the region separated from the inner wall surface of the container 1, it is not necessary that the container have a flat bottom as in prior art, which enhances flexibility in the shape of the container, allowing for enhancing flexibility in designing the detection device. For example, the predetermined region 16 is preferably separated from the inner wall surface of the container 1 in the range from several micrometers to several centimeters, in particular, in the range from several tens of micrometers to several millimeters. Additionally, it is preferable that the predetermined region 16 does not include the bottom of the container 1 and thus is a region separated from the bottom of the container 1. This is because movement of the composite particles 13 may be hindered at the bottom of the container 1 and a substance settled on the bottom other than the composite particles may make the detection difficult as noise.

In the example shown in FIG. 1, an illumination device 5 serves as the flow generating unit 2. More specifically, light radiated from the illumination device 5 heats the solution 14. As a result, the flow generating unit 2 (the illumination device 5) can heat the solution 14 to cause convection therein to generate a flow in the first direction 21 at least in part of the solution 14.

The magnetic field generating unit 3 generates in the solution 14 a magnetic field gradient for moving the composite particles 13 in a second direction 31 different from the first direction 21. The composite particles 13 are moved in the second direction 31 by the resultant of force in the first direction 21 and force caused by the magnetic field gradient. As the magnetic field generating unit 3, for example, a magnet or an electromagnet can be used.

The detection unit 4 includes an imaging unit 44 and a processing unit 45. The imaging unit 44 has the function of taking a picture to capture an image. As the imaging unit 44, for example, an image capturing device, such as a camera or a video camera for capturing still images or moving images, may be used. The processing unit 45 has the function of detecting composite particles from the captured images. As the processing unit 45, for example, a computer including a CPU and a memory can be used. The function of the processing unit 45 detecting composite particles from images captured by the imaging unit 44 is executed by the CPU in the processing unit 45 in accordance with a program prestored in the memory in the processing unit 45. The detection unit 4 detects the composite particles 13, which are the substance 11 to be measured to which the magnetic labeling substance 12 is bound, based on motion of particles in the predetermined detection region 16 in the solution 14. Illumination light 51 radiated from the illumination device 5 is reflected by a mirror 43 to illuminate the solution 14. As the illumination light 51, spatial light can be used. In other words, the illumination device 5 is a light source that radiates spatial light into the container 1. Spatial light (also referred to as “propagating light”) is ordinary light propagating in space rather than localized light, such as near-field light. More specifically, spatial light generally refers to light that does not include near-field light, which is rapidly attenuated at a position several hundreds of nanometers to several micrometers away from its source. In the present description also, it refers to light that does not include near-field light, i.e., light that is not rapidly attenuated at a position several hundreds of nanometers to several micrometers away from the interface between the container and the solution. Since the predetermined region 16 in the present description is a region separated several micrometers or more from the inner wall surface of the container 1, near-field light is not used in the predetermined region 16. Detection light 41 reflected by the composite particles 13 in the solution 14 enters the imaging unit 44 of the detection unit 4.

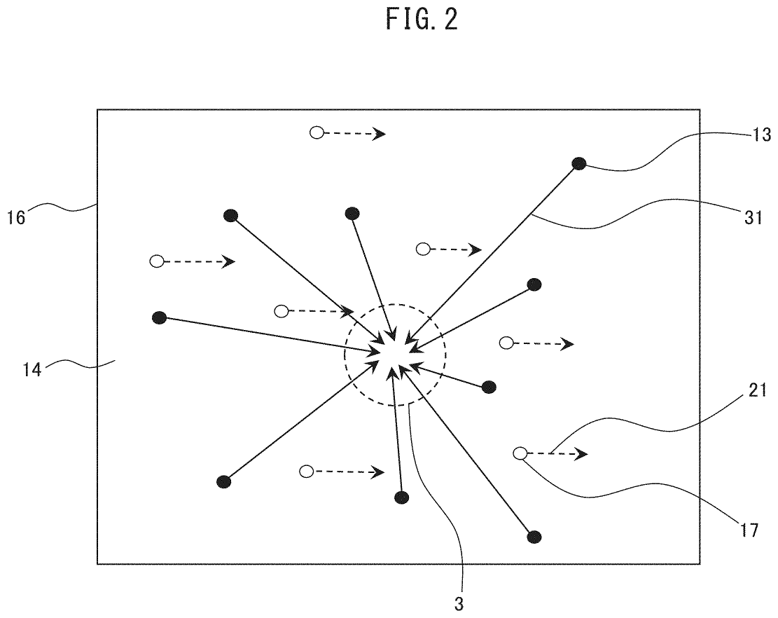

FIG. 2 shows the moving directions of the substance to be measured and other substances in the detection region in the solution detected by the detection device according to embodiment 1 of the present disclosure (an example image).

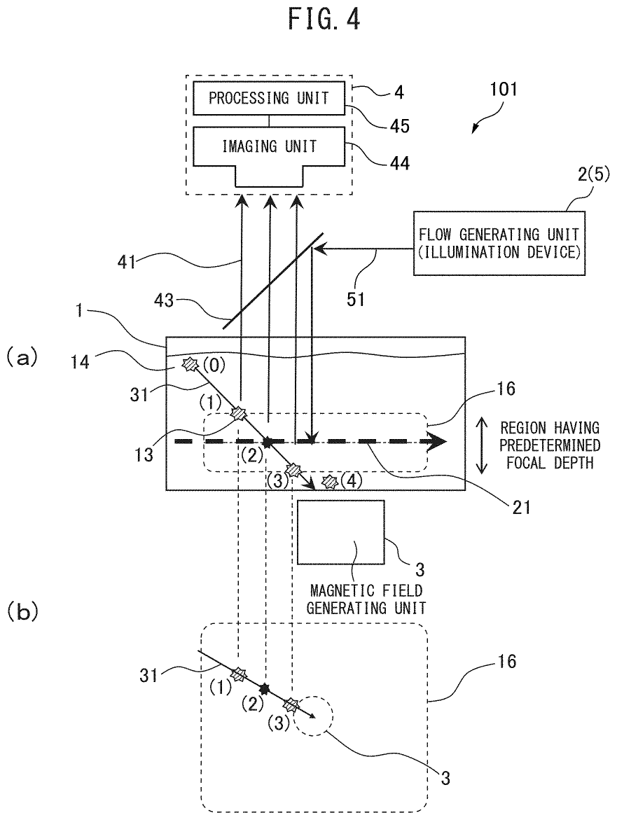

The magnetic labeling substance 12 binds specifically to the substance 11 to be measured. The solution 14 may contain other substances 17 that are not the substance 11 to be measured nor the magnetic labeling substance 12. The “other substances” are not the substance to be measured and include impurities. The magnetic labeling substance 12 does not bind to the other substances 17. As shown in FIG. 2, the composite particles 13, which are the substance 11 to be measured to which the magnetic labeling substance 12 is bound, are affected by the magnetic field gradient generated by the magnetic field generating unit 3, moving in the second direction 31 different from the first direction 21 in images captured by the imaging unit 44 of the detection unit 4. In contrast, the other substances 17, which do not include the magnetic labeling substance 12, do not follow the magnetic field gradient but move with the flow in the first direction 21. Thus particles moving in the second direction 31, the direction toward the magnetic field generating unit 3, are the composite particles 13, and the number of composite particles 13, i.e., that of particles of the substance to be measured can be detected by detecting the number of particles moving in the second direction 31. In FIG. 2, the arrows extending from the composite particles 13 and the other substances 17 schematically indicate the directions of motion of the respective particles, but the lengths of the arrows do not indicate the speeds of motion of the particles. The detection unit 4 can detect the composite particles 13, which are the substance 11 to be measured to which the magnetic labeling substance 12 is bound, based on the characteristic motion of particles to be measured. The detection unit 4 may detect the composite particles 13, based on the characteristic motion of particles to be measured and motion of the other substances 17 different from such characteristic motion in the predetermined detection region 16 in the solution 14. Details of the method for detecting the substance to be measured will be described below with reference to FIGS. 4 to 7.

The composite particles 13 is simultaneously acted on not only by force in the first direction 21 but also by force in a direction different from the first direction 21 caused by the magnetic field gradient. If only the force in a direction different from the first direction 21 caused by the magnetic field gradient acts on the composite particles 13, the other substances 17, which are not the targets for measurement, also move simultaneously by being pulled by the composite particles 13, which may result in erroneous detection of the number of particles. Thus the detection device according to the embodiment of the present disclosure makes force in two different directions, i.e., in the first direction 21 and a direction different from the first direction 21 act on the composite particles 13, allowing for separating the other substances 17 from the composite particles 13.

The magnetic field generating unit 3 may move the composite particles 13 in a second direction 31, which is identical to the first direction 21. In this case, the detection unit 4 can detect the composite particles 13, based on the speeds of motion of the composite particles 13 and the other substances 17. Even if the second direction 31, the direction of motion of particles caused by the magnetic field gradient, is identical to the first direction 21, the direction of motion of particles caused by the flow generating unit 2, the composite particles 13 including the magnetic labeling substance 12 move faster than the other substances 17, which do not include the magnetic labeling substance 12, due to the magnetic field gradient. Thus the composite particles 13 can be detected from obtained images, based on the fact that their speeds differ. If the second direction 31, the direction of motion of particles caused by the magnetic field gradient, is opposite to the first direction 21, the direction of motion of particles caused by the flow generating unit 2, the speeds and direction of motion of the composite particles 13 including the magnetic labeling substance 12 differ from those of the other substances 17, which do not include the magnetic labeling substance 12, due to the magnetic field gradient. Thus the composite particles 13 can be detected from obtained images.

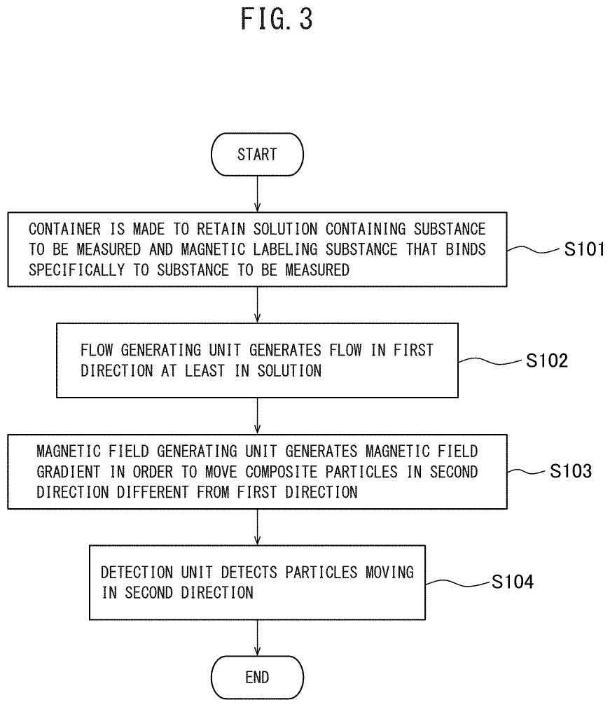

The following describes a method for detecting a substance to be measured according to embodiment 1 of the present disclosure. FIG. 3 shows a flowchart for explaining the steps of a method for detecting a substance to be measured according to embodiment 1 of the present disclosure. First, in step S101, the container 1 is made to retain the solution 14 containing the substance 11 to be measured and the magnetic labeling substance 12 that binds specifically to the substance 11 to be measured. It is not necessary that the magnetic labeling substance 12 bind to the substance 11 to be measured at the very moment when they are injected into the container 1.

Next, in step S102, the flow generating unit 2 generates a flow in the first direction 21 at least in the solution 14. In the example shown in FIG. 1, the illumination device 5 serves as the flow generating unit 2, as described above. For example, the flow of the solution 14 generated in the container 1 facilitates a reaction by which the magnetic labeling substance 12 binds to the substance 11 to be measured, thereby generating the composite particles 13.

Next, in step S103, the magnetic field generating unit 3 generates a magnetic field gradient in order to move the composite particles 13 in the second direction 31 different from the first direction 21.

Next, in step S104, the detection unit 4 detects particles moving in the second direction 31. More specifically, the imaging unit 44 of the detection unit 4 captures images of the detection region 16 in the solution 14, and the processing unit 45 executes a process for detecting the composite particles 13 and the other substances 17 (described below), using these captured images. The following describes the “method for detecting the composite particles 13,” which is divided into a “method for adjusting the focus of an image,” a “method for image processing by which the detection unit detects the composite particles from an obtained image,” and a “method for recognizing particles moving in obtained images.”

First, a method by which the detection unit 4 adjusts the focus of an image (the focal depth at capturing the image) will be described in detail. FIG. 4(a) shows a side view of the detection device 101 according to embodiment 1 of the present disclosure for explaining a trajectory of the substance to be measured in the detection device. FIG. 4(b) shows a top view of the detection region 16 viewed from the side of the detection unit 4 in FIG. 4(a).

The imaging unit 44 has the function of adjusting the focus, and can be set so as to have a predetermined focal depth in the detection region 16. FIG. 4(a) shows a composite particle 13 moving in the second direction 31 toward the bottom of the container 1 in the order of (1), (2), and (3). Assume that the imaging unit 44 is in focus best when the composite particle 13 is at the position of (2), and that the composite particle 13 can be recognized from the position of (1) closer to the surface of the solution 14 than (2) to the position of (3) closer to the bottom of the container 1 although it is not completely in focus. Such setting of the imaging unit 44 enables a composite particle 13 to be tracked from when it reaches the position of (1) until it reaches the position of (3) by following the magnetic field gradient, allowing for observing how it moves in the detection region 16. Thus, as shown in FIG. 4(b), the detection unit 4 can detect a composite particle 13 moving in the second direction 31 in the detection region 16. Additionally, if the composite particles 13 are first gathered, for example, by a magnetic field generating unit (not shown), at a portion inside the container 1 above the detection region 16, as shown at the position of (0) in FIG. 4(a), the composite particles 13 fall down through the detection region 16 toward the lower side of the detection region 16 by magnetic force caused by the magnetic field generating unit and gravity with the elapse of time, as shown at the position of (4) in FIG. 4(a). For this reason, substantially all the composite particles 13 can be counted by observing the detection region 16 for a predetermined time period.

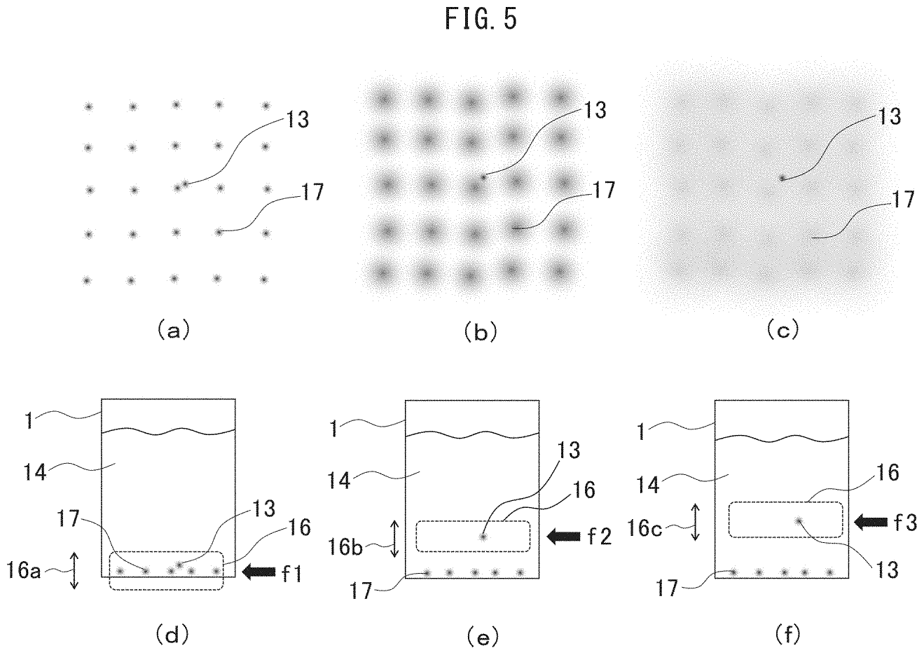

FIGS. 5(a) to (c) show plan views of images obtained at different focal depths by the detection device 101 shown in FIG. 4(a). FIGS. 5(a) to (c) schematically show an example in which the composite particles 13 and the other substances 17 are disposed in a grid-like pattern. However, in practice, the composite particles 13 and the other substances 17 are not necessarily disposed in a grid-like pattern. FIGS. 5(d) to (f) show side views of the container 1 of the detection device corresponding to FIGS. 5(a) to (c), respectively. As shown in FIG. 5(d), if the detection region 16 is a predetermined region 16a near the bottom of the container 1 and the imaging unit 44 is in focus at a position f1 near the bottom of the container 1, not only the composite particles 13 but also the other substances 17, which are not the targets for measurement, are in focus as shown in FIG. 5(a), which makes identification of the composite particles 13 difficult.

Thus it is preferable that the detection region 16 be set in a region 16b or 16c located a predetermined distance away from the bottom of the container 1, and that the focus f2 or f3 of the imaging unit 44 be set near the center of the corresponding region, as shown in FIG. 5(e) or (f). Such setting of the imaging unit 44 results in only the composite particles 13 in the region 16b or 16c being in focus and the other substances 17 at the bottom of the container 1, which are not the targets for measurement, being out of focus, as shown in FIG. 5(b) or (c), enabling the detection unit 4 to easily detect the composite particles 13.

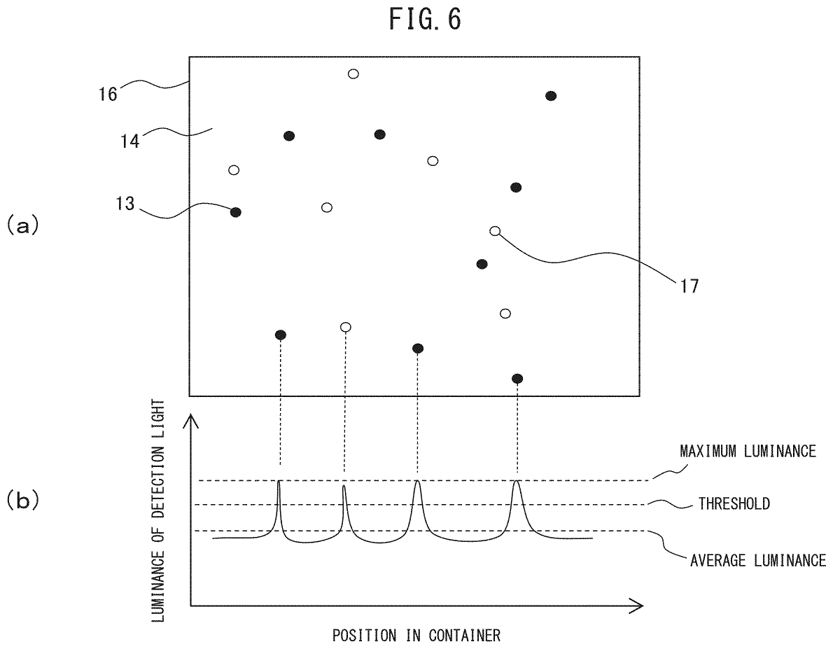

The following describes a method for image processing by which the detection unit detects the composite particles from an obtained image. FIG. 6(a) shows an image of the detection region 16 in the solution 14 captured by the imaging unit 44 of the detection unit 4 constituting the detection device according to embodiment 1 of the present disclosure, and FIG. 6(b) shows the luminance of detection light of the particles in the image of FIG. 6(a) obtained by image processing of the processing unit 45 of the detection unit 4. As shown in FIG. 6(b), using the intermediate point between the average luminance and the maximum luminance of the screen as a threshold, the processing unit 45 of the detection unit 4 can judge portions whose luminance exceeds the threshold to be particles in an image obtained by the imaging unit 44 of the detection unit 4. However, the threshold for judgment of particles is not limited to the one in this example and can be set as desired. Further, the imaging unit 44 can continuously capture images of the detection region 16 in the solution 14, and the processing unit 45 can continuously execute the process for detecting the composite particles 13, based on the images captured by the imaging unit 44.

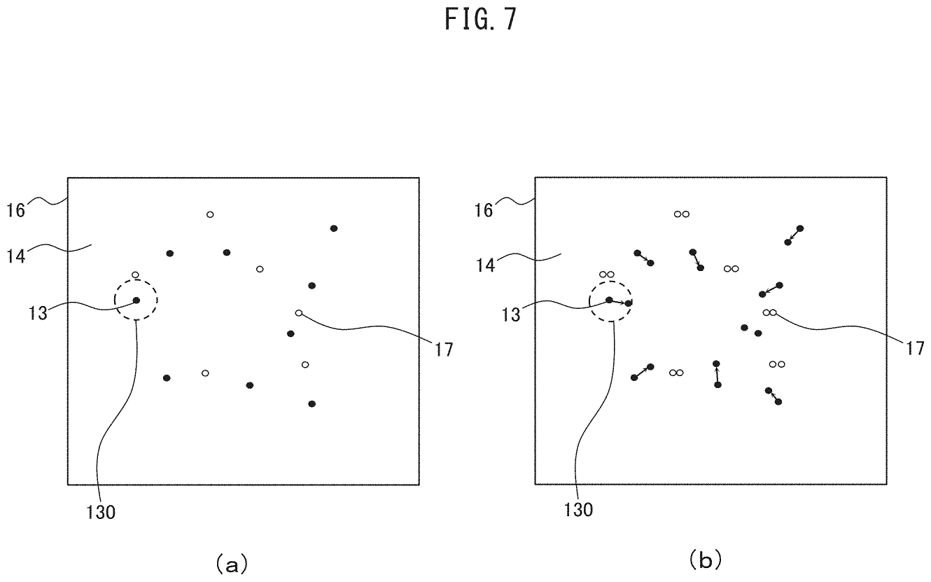

The following describes a method by which the detection unit 4 recognizes particles moving in obtained images. FIG. 7(a) shows an initial image of the detection region 16 in the solution 14 captured by the imaging unit 44 of the detection unit 4 constituting the detection device according to embodiment 1 of the present disclosure. FIG. 7(b) shows superposition of the initial image and an image obtained after the elapse of a predetermined time period from the capture of the initial image. The following describes an example in which the imaging unit 44 captures moving images composed of multiple frames and the processing unit 45 executes image processing, using the frames, which are individual still images constituting the captured moving images. The maximum moving speed of particles is tentatively set, and a moving distance 130 that the same particle would travel between two successive frames is set. These frames are the initial image and the image obtained after the elapse of a predetermined time period from the capture of the initial image. Next, it is judged that a target particle in the first frame in FIG. 7(a) and a particle in the next frame in FIG. 7(b) located within the moving distance 130 of the target particle and having a coordinate closest to that of the target particle are probably the same particle. A similar process is applied to the next frame, and for example, particles in five or more successive frames judged to be the same particle are preferably registered as a single particle in a database. Such a process for recognizing movement may be executed on multiple particles in multiple frames to create a database of particle coordinates. The detection unit 4 detects the composite particles 13 from among the detected particles, based on motion of the detected particles.

When fluorescent light is not used, all the substances in the sample solution that scatter light are recorded in the images, and thus if there are particles receiving force caused by the magnetic field gradient other than the composite particles 13, these particles are also recorded in the images. For this reason, it is necessary to separate the particles recorded in the images.

More specifically, a process is necessary for excluding a separate magnetic labeling substance 12 and particles of impurities or other substances to which the magnetic labeling substance 12 is nonspecifically bound, using the speeds and directions of movement vectors. This process will be described below.

First, a separate magnetic labeling substance 12 will be considered. A separate magnetic labeling substance 12 moves faster than a composite particle in the same magnetic field gradient because it does not have an extra load (a counterpart to form a composite particle) that would exist if it were a composite particle. It can be separated by setting a threshold at the known maximum moving speed of the composite particles and excluding target particles having a speed greater than the threshold. Since the moving speed varies depending on the magnitude of the magnetic field gradient, i.e., the place in the detection region, it is necessary to set thresholds for respective places beforehand by calculation or measurement and to store them in the processing unit 45.

The magnetic labeling substance 12 nonspecifically bound to impurities can be separated by setting a threshold at the known minimum speed of the composite particles 13 and excluding target particles having a speed less than the threshold. However, theoretically, if the magnetic labeling substance 12 has properties (the size, molecular weight, and surface state) similar to those of the substance 11 to be measured, the specificity of binding to the substance 11 to be measured needs to be set sufficiently high as necessary.

The detection unit of the detection device according to embodiment 1 of the present disclosure may detect the composite particles, based on the moving speeds of objects moving in the predetermined region of the container. The moving directions and speeds of particles are determined from the database of particle coordinates created as described above. More specifically, when the magnetic field generating unit 3 is disposed at the center of the screen as shown in FIG. 2, the particles moving in the second direction 31 toward the center of the screen can be recognized as the composite particles 13 and the number of particles can be counted. Since the speeds of the composite particles 13 increase as they approach the center, whether the speed increases depending on the distance from the center can be added as a criterion. Since force caused by the flow in the first direction 21 acts also on the composite particles 13, trajectories described by the composite particles 13 are not straight lines in some cases. In these cases, it is preferable to compensate for the effect of the flow to determine the trajectories. For example, when rotation of the container 1 is used as external force, the other substances 17 describe circular trajectories and the composite particles 13 spiral trajectories. In this way, the composite particles 13, which are targets for detection, and the other substances 17 may be identified, based on the difference between the shapes of the trajectories described by particles. Since the speeds of the composite particles 13 increase as they approach the center, whether the speed increases depending on the distance from the center can be added as a criterion.

The following describes a device for detecting a substance to be measured according to modified example 1 of embodiment 1 of the present disclosure. In the above embodiment is shown the example in which the flow generating unit 2 heats the solution 14 to cause convection therein to generate a flow in the first direction 21 at least in part of the solution 14, but the invention is not limited to this example. More specifically, the flow generating unit 2 may rotate the container 1 to generate a flow in the first direction 21 at least in part of the solution 14.

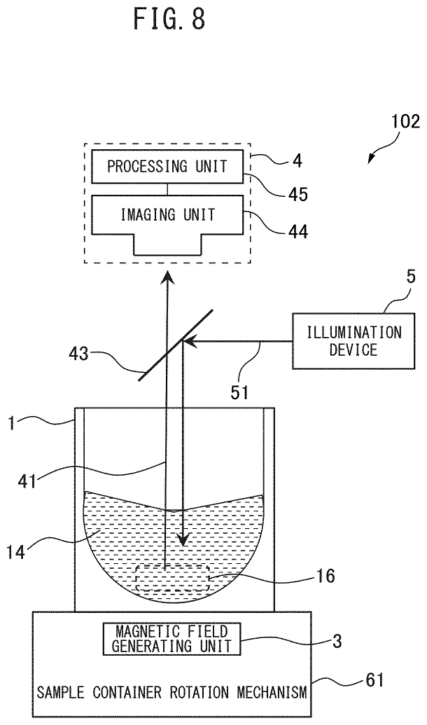

FIG. 8 shows the configuration of a device 102 for detecting a substance to be measured according to modified example 1 of embodiment 1 of the present disclosure. In the example shown in FIG. 8, a sample container rotation mechanism 61 functions as the flow generating unit. The container 1 is placed on the sample container rotation mechanism 61 and rotated by the sample container rotation mechanism 61 to generate a flow in a first direction in the solution 14 by centrifugal force. The magnetic field generating unit 3 may be incorporated in the sample container rotation mechanism 61. Details of a method for determination will be described below.

The following describes a device for detecting a substance to be measured according to modified example 2 of embodiment 1 of the present disclosure. The detection device according to modified example 2 is characterized in that the flow generating unit stirs the solution to generate a flow in a first direction at least in part of the solution.

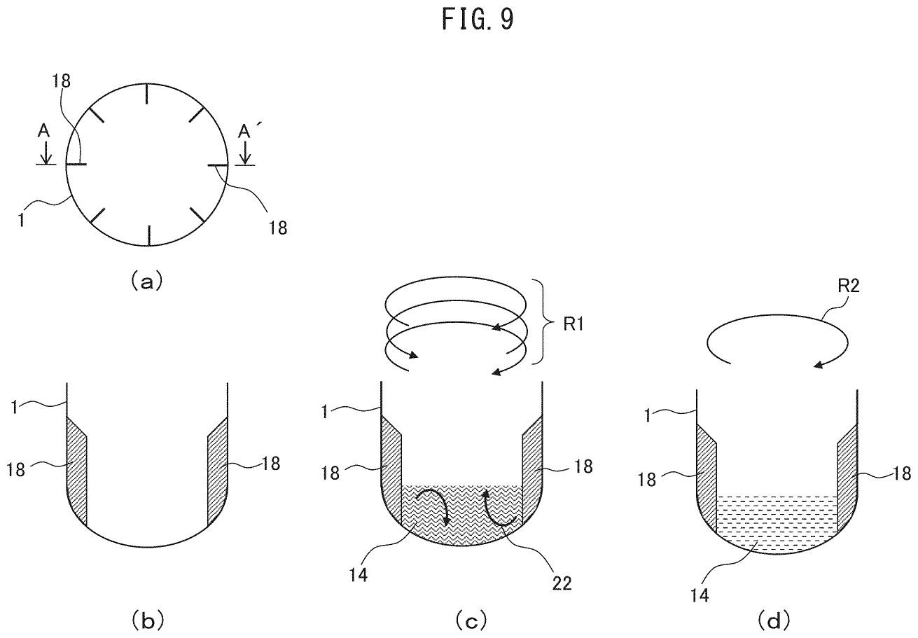

FIGS. 9(a) to (d) show the configuration of a stirrable container used in a device for detecting a substance to be measured according to modified example 2 of embodiment 1 of the present disclosure. FIG. 9(a) shows a plan view of the stirrable container, and FIGS. 9(b) to (d) cross-sectional views taken along line A-A′ in FIG. 9(a). FIG. 9(c) shows how the container rotates at stirring, and FIG. 9(d) shows how the container rotates at detection of the substance to be measured.

As shown in FIGS. 9(a) and (b), the rotatable container 1 is preferably equipped with fins 18 for stirring on its inner wall. At stirring, the container 1 preferably repeats rotation and counter rotation multiple times, as indicated by R1 in FIG. 9(c). Stirring can generate a turbulent flow 22 in the solution 14, facilitating a reaction between particles dispersed in the solution 14. Additionally, at detection by image processing, the container 1 can be rotated at a constant speed, as indicated by R2 in FIG. 9(d), applying centrifugal force to the particles in the solution 14 as external force.

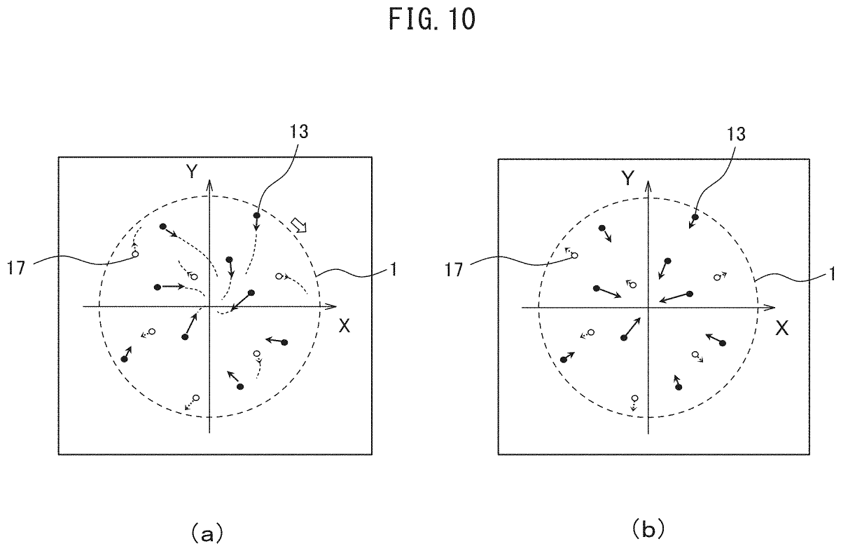

FIG. 10(a) shows, with arrows, the positions and motion of particles at a certain time for the case that the container is rotated. The black and white dots indicate the composite particles 13 and the other substances 17, respectively. The broken line indicates the outer portion of the container 1. Since the magnetic field generating unit 3 is disposed at the center of the container 1 (see FIG. 8), clockwise rotation of the container 1 indicated by a solid-white arrow causes the composite particles 13 to be rotated and drawn toward the center of the container 1, where the magnetic field gradient is greatest, to describe spiral trajectories as indicated by dotted lines. In contrast, the other substances 17, on which centrifugal force in the direction from the center of rotation of the container 1 toward the outside acts, describe spiral trajectories toward the outside as indicated by dotted lines. When the container 1 is rotated, the magnetic field and the rotation rate are set so that the magnetic force may constantly exceed the centrifugal force in a region in the sample solution in the container 1, allowing for drawing the composite particles 13 contained in the container 1 toward the center of the container 1. In FIG. 10(a), the dotted lines represent the trajectories only for some of the particles that are representatives.

The following describes a method for determination by which the composite particles and the other substances are separated using movement vectors of particles. First, a “rotation process” is performed for removing motion of particles arising from rotation of the container 1. When the rotation rate of the container 1 is known, the obtained images are rotated opposite to the rotating direction of the container 1. FIG. 10(b) shows the positions and motion of the particles (the composite particles 13 and the other substances 17) after the rotation process. As a result of canceling out the motion of the particles caused by rotation, the particles move only in the radial direction of the container 1. The composite particles 13 receive centrifugal force and magnetic force stronger than it, moving toward the center of the container 1. In contrast, the other substances 17 receive only centrifugal force, moving from the center of the container 1 toward the outside. Thus the composite particles 13 can be detected according to the moving directions of the particles. More specifically, the composite particles 13, hence the substance to be measured, can be detected by detecting particles moving toward the center of the container 1.

As a more convenient method for determination, changes in the distance from the origin of XY coordinates of the particles can be used in FIG. 10(a). It can be determined that a target particle is a composite particle 13, if the distance from the origin decreases after the elapse of a certain time period, and that it is another substance 17, if the distance increases. In this case, the rotation process is not necessary because it does not change the distances of particles from the origin.

The following describes a device for detecting a substance to be measured according to modified example 3 of embodiment 1 of the present disclosure. In the above embodiment is shown the example in which the detection unit 4 placed above the container 1 is used to detect the composite particles, but the invention is not limited to this example. The composite particles may be detected from the side surface of the container in parallel with the horizontal direction.

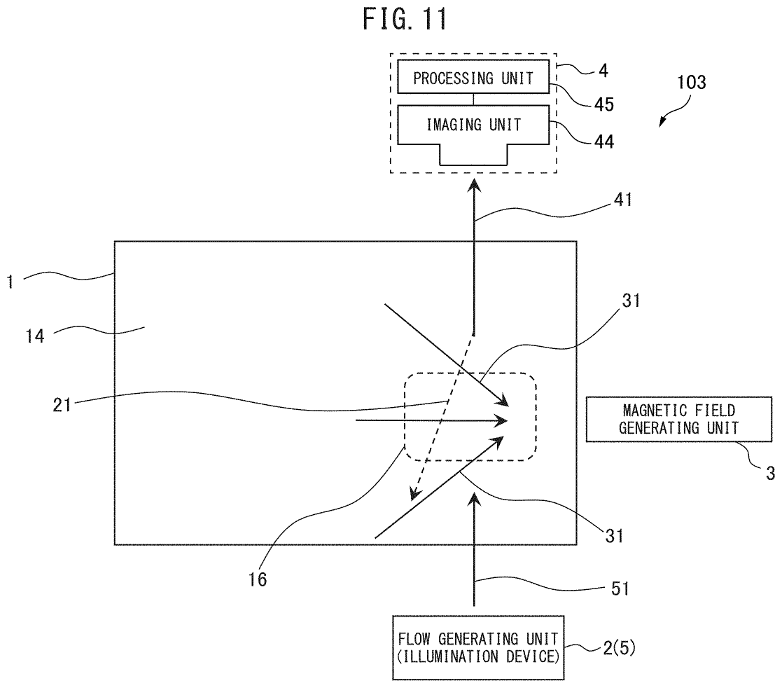

FIG. 11 shows the configuration of a device 103 for detecting a substance to be measured according to modified example 3 of embodiment 1 of the present disclosure. FIG. 11 shows the configuration of the detection device 103 as viewed from a direction orthogonal to the illumination light 51 and the detection light 41. The illumination light 51 from the illumination device 5 generates a flow in a first direction 21 in the detection region 16. Thus the illumination device 5 serves as the flow generating unit 2. The composite particles follow the magnetic field gradient generated by the magnetic field generating unit 3 to move in a second direction 31. FIG. 11 shows the example in which the detection unit 4 and the illumination device 5 are disposed on opposite sides, but they may be disposed on the same side.

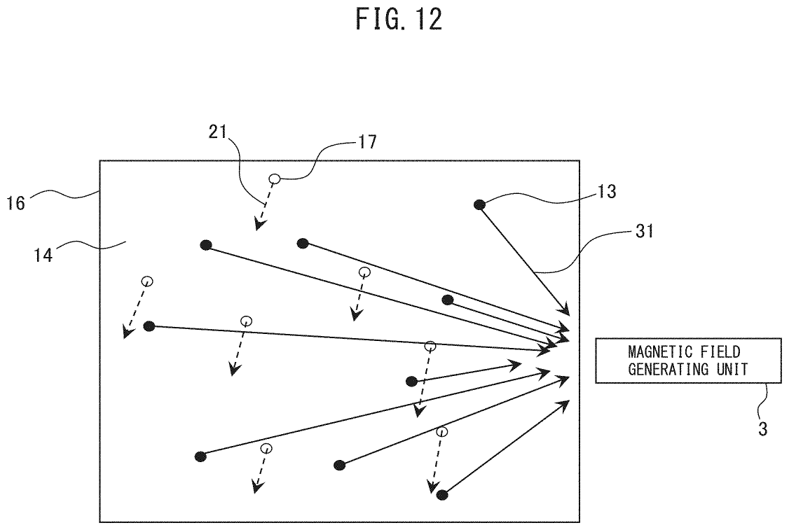

FIG. 12 shows the moving directions of the substance to be measured and other substances in the detection region in the solution detected by the detection device according to modified example 3 of embodiment 1 of the present disclosure. The composite particles 13 follow the magnetic field gradient generated by the magnetic field generating unit 3 to move in the second direction 31. In contrast, the other substances 17, which are not the targets for detection and to which the magnetic labeling substance is not bound, move in the first direction 21 different from the second direction 31 with the flow generated by the flow generating unit 2. The composite particles 13 can be detected by detecting particles moving in the second direction 31.

The detection device according to modified example 3 of embodiment 1 of the present disclosure can detect the moving composite particles 13 in the detection region 16 in a direction substantially orthogonal to the direction of the magnetic field gradient, allowing for detecting the same composite particle for a longer time than when observing in a direction substantially the same as the direction of the magnetic field gradient.

According to the detection device and method according to embodiment 1, the substance to be measured can be easily detected by detecting the composite particles that are the substance to be measured to which the magnetic labeling substance is bound, as described above.

### Embodiment 2

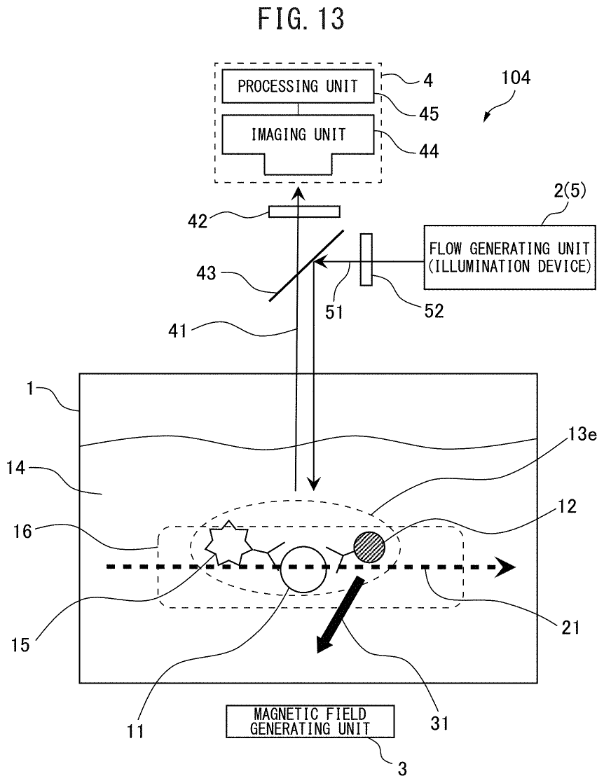

The following describes a device for detecting a substance to be measured according to embodiment 2 of the present disclosure. FIG. 13 shows the configuration of a device 104 for detecting a substance to be measured according to embodiment 2 of the present disclosure. The detection device 104 according to embodiment 2 differs from the detection device 101 according to embodiment 1 in that composite particles 13e further include a fluorescent labeling substance 15 and that the detection unit 4 detects particles to which the fluorescent labeling substance 15 is bound. The other components of the detection device 104 according to embodiment 2 are identical to those of the detection device 101 according to embodiment 1, and thus detailed description thereof is omitted.

The container 1 retains a solution 14 containing a substance 11 to be measured as well as a magnetic labeling substance 12 and a fluorescent labeling substance 15 that bind specifically to the substance 11 to be measured. The magnetic labeling substance 12 and the fluorescent labeling substance 15 preferably bind to all the substance 11 to be measured in the solution 14 to form composite particles 13e.

It is not necessary that the magnetic labeling substance 12 and the fluorescent labeling substance 15 bind to the substance 11 to be measured at the very moment when they are injected into the container 1. More specifically, for example, a flow of the solution 14 generated in the container 1 may facilitate a reaction by which the magnetic labeling substance 12 and the fluorescent labeling substance 15 bind to the substance 11 to be measured, thereby generating the composite particles 13e.

Illumination light 51 radiated from the illumination device 5 passes through an illumination-side optical filter 52, and is reflected by a mirror 43 to illuminate the solution 14. As the illumination light 51, spatial light can be used. Detection light 41 reflected by the composite particles 13e and substances 17 other than the substance to be measured in the solution 14 enters the detection unit 4 through a detection-side optical filter 42. The illumination-side optical filter 52 passes light having wavelengths such that it illuminates and thereby excites the fluorescent labeling substance 15 so as to emit fluorescent light, but does not pass light having the other wavelengths. The detection-side optical filter 42 passes the fluorescent light emitted from the fluorescent labeling substance 15, but does not pass light having the other wavelengths.

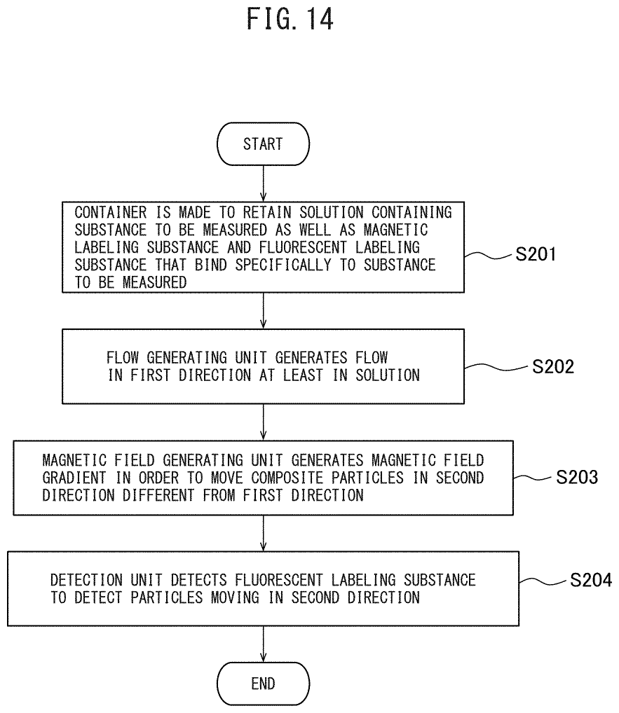

FIG. 14 shows a flowchart for explaining the steps of a method for detecting a substance to be measured according to embodiment 2 of the present disclosure. The detection method according to embodiment 2 differs from the detection method according to embodiment 1 in that the composite particles 13e further include a fluorescent labeling substance 15 and that particles to which the fluorescent labeling substance 15 is bound are detected.

Of fluorescent labeling substances 15, some bind specifically to the substance 11 to be measured, and others do not. The present embodiment describes the case in which a fluorescent labeling substance 15 that binds specifically to the substance 11 to be measured is used.

First, in step S201, the container 1 is made to retain the solution 14 containing the substance 11 to be measured as well as the magnetic labeling substance 12 and the fluorescent labeling substance 15 that bind specifically to the substance 11 to be measured.

Next, in step S202, the flow generating unit 2 generates a flow in the first direction 21 at least in the solution 14. In the example shown in FIG. 13, the illumination device 5 serves as the flow generating unit 2. The flow generated in the solution 14 yields the composite particles 13e that are the substance 11 to be measured to which the magnetic labeling substance 12 and the fluorescent labeling substance 15 are bound.

Next, in step S203, the magnetic field generating unit 3 generates a magnetic field gradient in order to move the composite particles 13e in the second direction 31 different from the first direction 21.

Next, in step S204, the detection unit 4 detects the fluorescent labeling substance 15 to detect particles moving in the second direction 31 to which the fluorescent labeling substance 15 is bound.

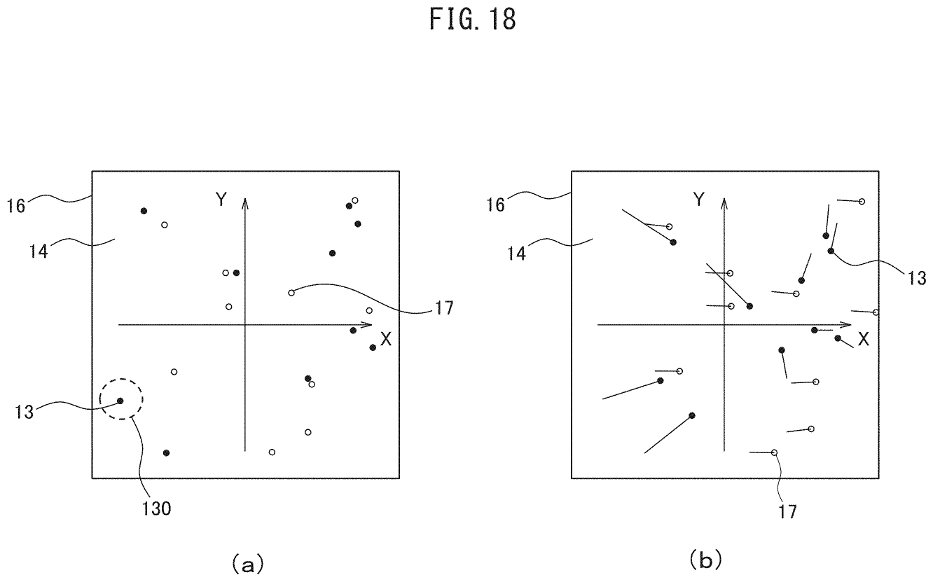

FIG. 18(a) shows an initial image of the detection region 16 in the solution 14 captured by the imaging unit 44 of the detection unit 4 constituting the detection device according to embodiment 2 of the present disclosure. FIG. 18(b) shows an image obtained after the elapse of a predetermined time period from the capture of the initial image. The black and white dots in FIGS. 18(a) and (b) indicate the composite particles 13 and the other substances 17, respectively. The line segments in FIG. 18(b) represent the trajectories of the particles from a certain time (e.g., at the capture of the initial image) until the elapse of a predetermined time period, and thus indicate the speeds and directions of movement of the particles. They will be referred to as movement vectors.

A method for separating the composite particles and the other substances will be specifically described, using the defined movement vectors. The following description relates to the case in which fluorescent light is used, but can also be applied similarly to the case in which fluorescent light is not used.

When fluorescent light is used, the other substances to be separated are particles that are not the composite particles 13 and that do not receive force in the second direction 31 caused by the magnetic field gradient. The other substances to be separated are, for example, a separate fluorescent labeling substance 15 that is not bound to any particle and particles of substances, such as impurities, that are not the substance to be measured and to which the fluorescent labeling substance 15 is bound.

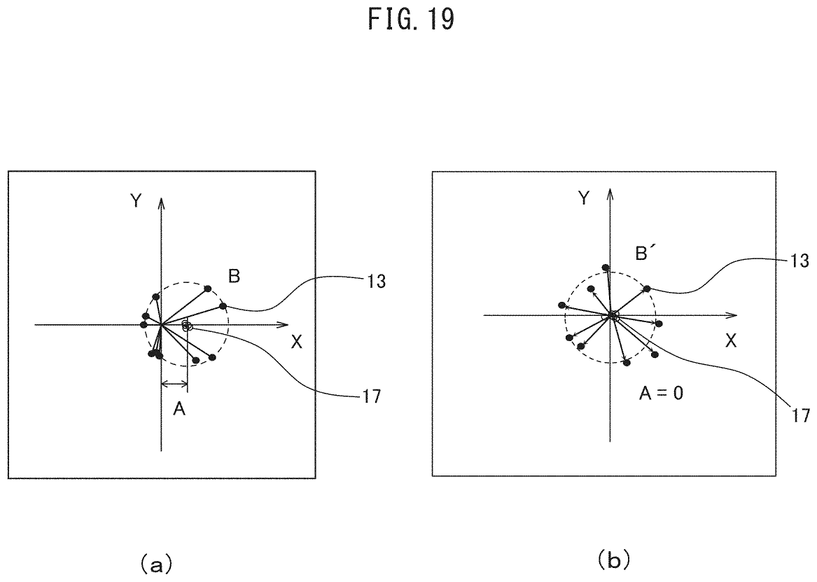

FIGS. 19(a) and 19(b) are plots of movement vectors from various viewpoints in which the initial points of the movement vectors are disposed at the origin of XY coordinates, the vectors are represented as line segments, and circular marks of particles are disposed at the final points of the vectors.

In FIG. 19(a), since the other substances 17 indicated by white dots receive force corresponding to the flow, i.e., the force in the first direction 21 (see FIG. 13), their movement vectors are concentrated on the right of the origin in the XY plane. In other words, it suggests that the speeds and directions of movement of the other substances 17 are substantially the same regardless of the positions of the other substances 17 in the XY plane. Arrow A is a representative of the movement vectors of the other substances 17.

The composite particles 13 receive the force in the second direction 31 (see FIG. 13) caused by the magnetic field gradient, but the magnitude and direction of the magnetic field gradient vary depending on the positions of the composite particles 13. For this reason, their vectors point in various directions from the origin of XY coordinates like movement vectors B indicated by line segments in FIG. 19(a). However, their final points are distributed on a circle having a substantially constant radius, as indicated by a dotted line in the figure. More specifically, the final points of the composite particles 13 are distributed on a circle having a radius in a predetermined range. This is because the composite particles 13 move toward the center of the magnet, where the magnetic field gradient is greatest, wherever they are located.

The reason the center of the circle is deviated from the origin is that both the force in the first direction 21 and the force in the second direction 31 act on the composite particles 13. In other words, the resultant of movement vector A and a movement vector caused by the force of the magnetic field gradient is movement vector B.

If the force in the first direction 21 is zero, the center of the circle of movement vectors B′ will agree with the origin, as shown in FIG. 19(b). In contrast, movement vector A will be zero because the other substances 17 will stand still.

By the above technique, the composite particles and the other substances can be separated, using the speeds and directions of movement of particles. The steps thereof are summarized as follows.

1) Successively determine vectors of movement of the particles at a certain time and after the elapse of a certain time period, as shown in FIG. 18(b), thereby creating a database of movement vectors.

2) Judge particles whose movement vectors are concentrated near the center as shown in FIG. 19(a) and vary little with the passage of time not to be the composite particles 13, using the database of movement vectors, and exclude them.

3) Judge particles whose movement vectors are in a circle centered at the final point of movement vector A as shown in FIG. 19(a) to be candidates for the composite particles, using the database of movement vectors.

4) Determine that candidates for the composite particles having movement vectors whose magnitudes vary with the passage of time and whose directions are unchanged are the composite particles 13, and count the number of particles of the candidates.

The number of composite particles can be counted from successively obtained images by the processing unit 45 executing the above process.

The time intervals at which the movement vectors are determined can be adjusted depending on the moving speeds of the particles and the frame rate of image capturing by a camera or other devices.

According to the detection device and method according to embodiment 2 of the present disclosure, particles smaller than the composite particles 13 in embodiment 1 can be detected by detecting particles to which the fluorescent labeling substance 15 is bound.

When a fluorescent labeling substance 15 that does not bind specifically to the substance 11 to be measured is used, the fluorescent labeling substance 15 may bind to the other substances 17. Even if the fluorescent labeling substance 15 is bound to the other substances 17, the composite particles can be distinguished from the other substances 17 to which the fluorescent labeling substance 15 is bound, and detected, based on the difference in motion. Further, there may be a fluorescent labeling substance 15 that is not bound to particles of the substance 11 to be measured, in the container 1. Even if there is a fluorescent labeling substance 15 that is not bound to particles of the substance 11 to be measured, the detection unit 4 can distinguish the composite particles from such a fluorescent labeling substance 15, and detect them, based on the difference in motion.

In the detection device and method according to embodiment 2, the region in the detection region 16 irradiated by the illumination device 5 with the illumination light 51 is preferably set so as to avoid the region of the magnetic field generating unit 3.

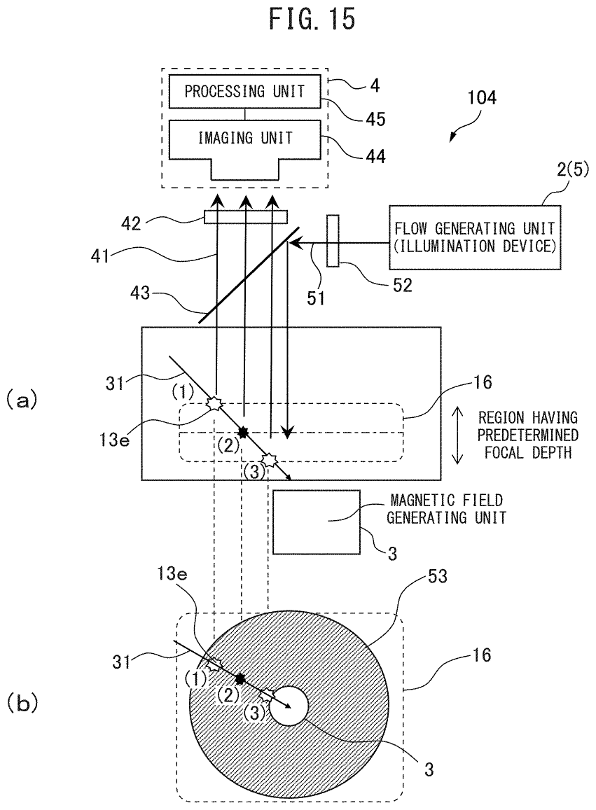

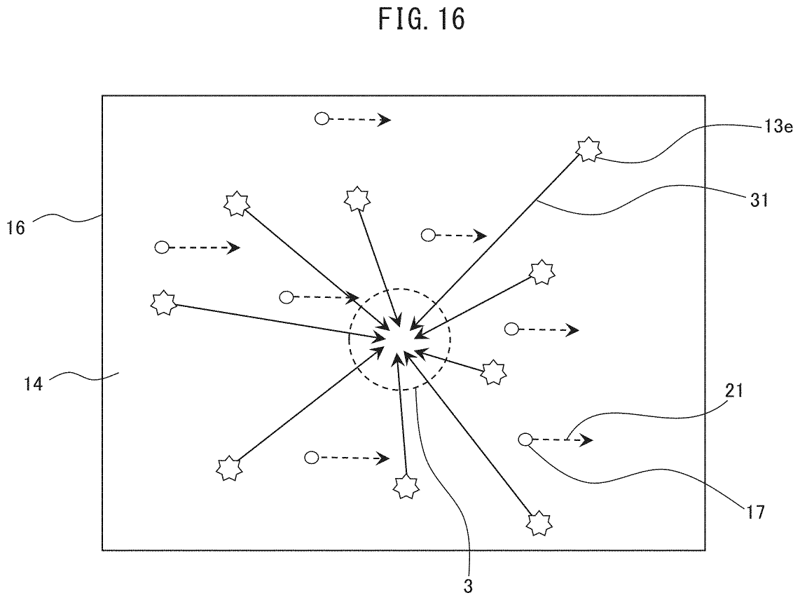

FIG. 15(a) shows a side view of the detection device 104 according to embodiment 2 of the present disclosure for explaining a trajectory of the substance to be measured in the detection device 104. FIG. 15(b) shows a top view of the detection region viewed from the side of the detection unit in FIG. 15(a). FIG. 16 shows the moving directions of the substance to be measured and other substances in the detection region in the solution detected by the detection device according to embodiment 2 of the present disclosure. The composite particles 13e to which the fluorescent labeling substance 15 is bound are drawn near the magnetic field generating unit 3 by the magnetic field gradient generated by the magnetic field generating unit 3. Since the illumination light 51 causes the composite particles 13e to emit fluorescent light, the aggregated composite particles 13e emit strong light, which may affect detection of the composite particles 13e in the other area in the detection region 16. Thus an illuminated region 53 that does not include an area around the magnetic field generating unit 3 is preferably set as the region irradiated with the illumination light 51.

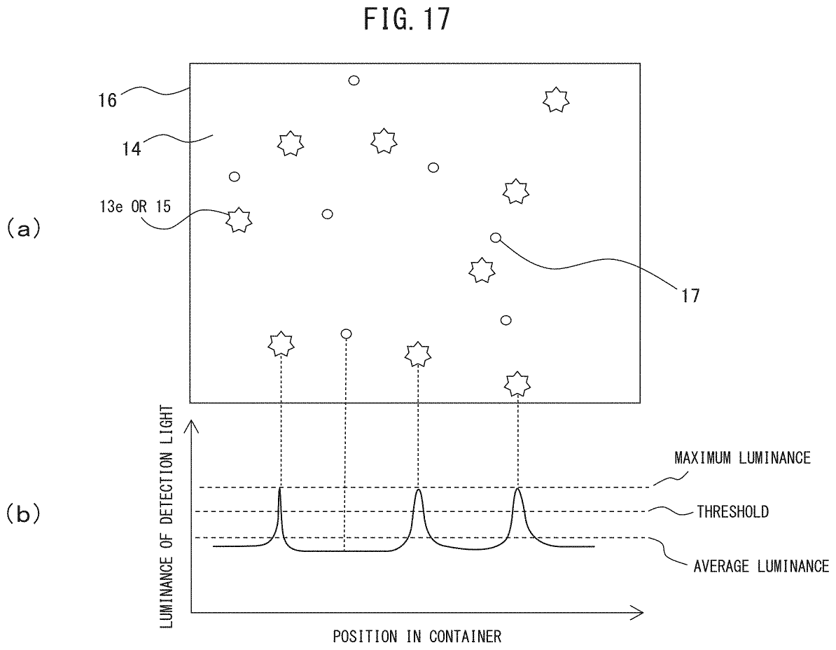

The following describes a method for image processing by which the detection unit detects the composite particles from an obtained image. FIG. 17(a) shows an image of the detection region in the solution captured by the imaging unit of the detection unit constituting the detection device according to embodiment 2 of the present disclosure, and FIG. 17(b) shows the luminance of detection light of the particles in the image of FIG. 17(a) obtained by image processing of the processing unit of the detection unit. The “other substances 17” are not shown in the image in practice, but are shown in FIG. 17(a) for convenience of explanation. The “other substances 17” represent particles to which the fluorescent labeling substance 15 is not bound. As shown in FIG. 17(b), the processing unit 45 of the detection unit 4 detects, by image processing, particles to which the fluorescent labeling substance 15 is bound, such as the composite particles 13e, and the fluorescent labeling substance 15 that is not bound to any substance, in the image captured by the imaging unit 44 of the detection unit 4. More specifically, using the intermediate point between the average luminance and the maximum luminance of the screen as a threshold, particles whose luminance exceeds the threshold can be judged to be the fluorescent labeling substance 15 or particles to which the fluorescent labeling substance 15 is bound. However, the threshold for judgment of the fluorescent labeling substance 15 or particles to which the fluorescent labeling substance 15 is bound is not limited to the one in this example and can be set as desired.

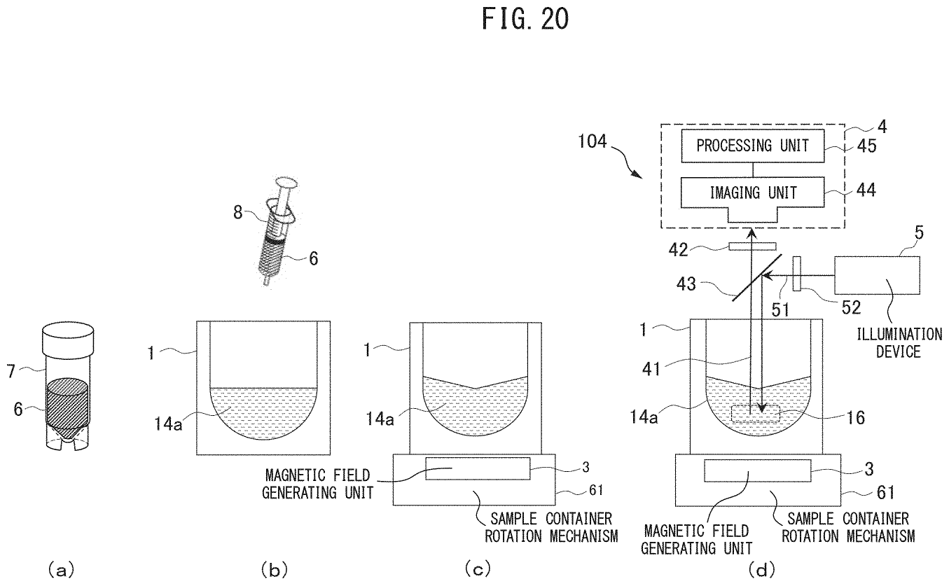

The following describes two specific examples of the detection method performed by the detection device according to embodiment 2 of the present disclosure. The first example is a detection method with a fluorescent labeling substance. FIGS. 20(a) to (d) show the steps of measurement for the case that a fluorescent labeling substance is used in the detection method according to embodiment 2 of the present disclosure.

First, as shown in FIG. 20(a), 0.5 [ml] of saliva 6 is collected in a sampling bottle 7. Next, as shown in FIG. 20(b), the saliva 6 is filtered with a syringe 8, and the filtered saliva 6 is added to the container 1 containing a solution with the fluorescent labeling substance 15 and the magnetic labeling substance 12 to make a solution 14a. The syringe 8 may have a filter for removing foreign substances (dust) larger than bacteria and fungi.

Next, as shown in FIG. 20(c), the solution 14a is stirred by the sample container rotation mechanism 61 to facilitate a reaction to form composites. Next, as shown in FIG. 20(d), a small magnet, which is the magnetic field generating unit 3, is brought nearer to the bottom of the container 1 while the solution 14a is rotated at a constant speed by the sample container rotation mechanism 61, concentrating the composite particles into a single point at the bottom of the container 1. At this time, the composite particles move toward the center of the container 1 while describing spiral trajectories due to stirring. This state is captured by the detection unit 4, and the composite particles are detected by image recognition as in FIGS. 5 to 7.

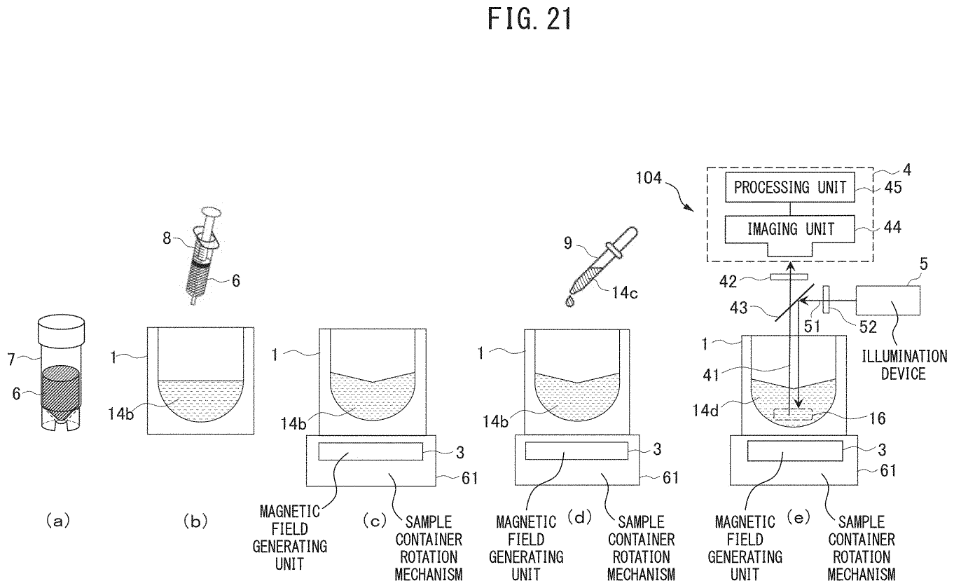

The second example is a detection method in which fluorescent staining is performed. FIGS. 21(a) to (e) show the steps of measurement for the case that fluorescent staining is performed in the detection method according to embodiment 2 of the present disclosure.

First, as shown in FIG. 21(a), 0.5 [ml] of saliva 6 is collected in a sampling bottle 7. Next, as shown in FIG. 21(b), the saliva 6 is filtered with a syringe 8, and the filtered saliva 6 is added to the container 1 containing a solution with a fluorescent stain solution (0.5 [ml]) to make a solution 14b.

Next, as shown in FIG. 21(c), the solution 14b is stirred by the sample container rotation mechanism 61 to facilitate staining. Next, as shown in FIG. 21(d), a solution 14c containing a magnetic labeling substance is added to the solution 14b to make a solution 14d, which is stirred by the sample container rotation mechanism 61 to facilitate formation of composite particles. Next, as shown in FIG. 21(e), a small magnet, which is the magnetic field generating unit 3, is brought nearer to the bottom of the container 1 while the solution 14d is rotated at a constant speed by the sample container rotation mechanism 61, concentrating the composite particles into a single point at the bottom of the container 1. At this time, the composite particles move toward the center of the container 1 while describing spiral trajectories due to stirring. This state is captured by the detection unit 4, and the composite particles are detected by image recognition as in FIGS. 5 to 7.

The values shown in the above are merely examples, and the invention is not limited thereto.

### Embodiment 3

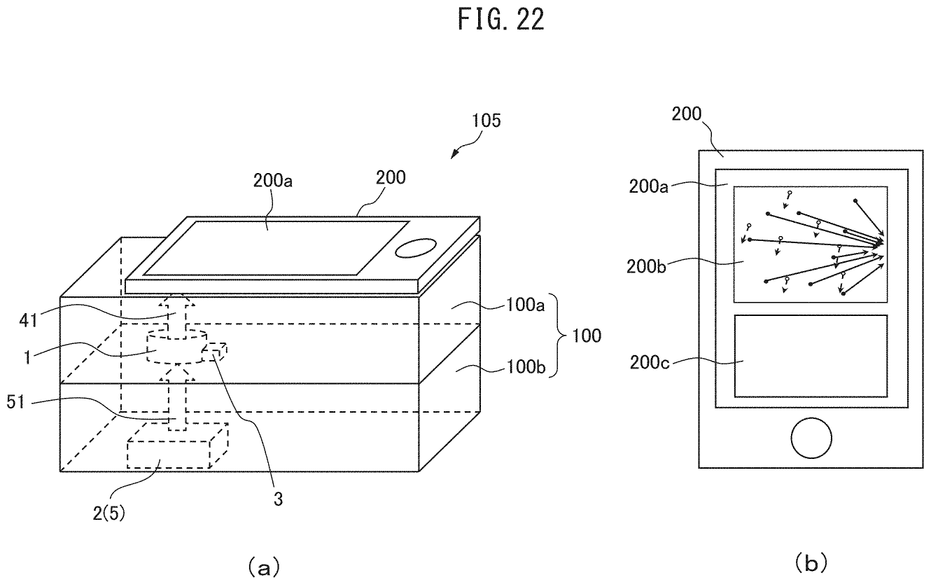

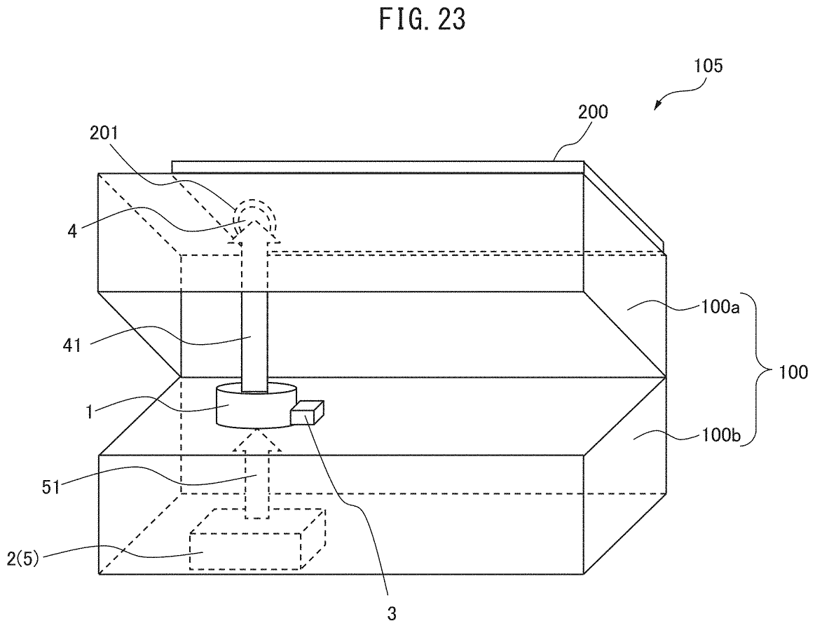

The following describes a device for detecting a substance to be measured according to embodiment 3 of the present disclosure. FIG. 22(a) shows a perspective view of a device 105 for detecting a substance to be measured according to embodiment 3 of the present disclosure. FIG. 22(b) shows an example of a display screen of a portable device for the case that the portable device is used as the detection device according to embodiment 3 of the present disclosure. FIG. 23 shows a perspective view of the detection device 105 according to embodiment 3 of the present disclosure in which a measurement housing is open.

The detection device 105 according to embodiment 3 of the present disclosure is characterized by using a portable device 200, such as a smartphone, to detect the substance to be detected. The container 1, the magnetic field generating unit 3, and the illumination device 5 are housed in a measurement housing 100. The measurement housing 100 is composed of an upper housing 100a and a lower housing 100b. The illumination device 5 is housed in the lower housing 100b. The container 1 is placed on the upper surface of the lower housing 100b. The magnetic field generating unit 3 is disposed on the side surface of the container 1. The portable device 200 is placed on the upper surface of the upper housing 100a, which has an opening 201 so that detection light 41 can enter the detection unit 4, which is, for example, a camera of the portable device 200. The illumination device 5 irradiates the container 1 with illumination light 51 from below, and the detection light 41 enters the detection unit 4 of the portable device 200. Since the container 1 is heated by the illumination light 51, the illumination device 5 serves as the flow generating unit 2.

The measurement principle of the detection device 105 according to embodiment 3 of the present disclosure is the same as that of the detection device 101 according to embodiment 1. Images captured by the detection unit 4 of the portable device 200 can be displayed in an image display area 200b in a display 200a of the portable device 200. Data analyzed from the obtained images, such as the number and moving speeds of particles of the substance to be measured, can be displayed in a data display area 200c in the display 200a. The substance to be measured can be more conveniently detected by detection of images and execution of image processing with a portable device, as in the embodiment of the present disclosure.

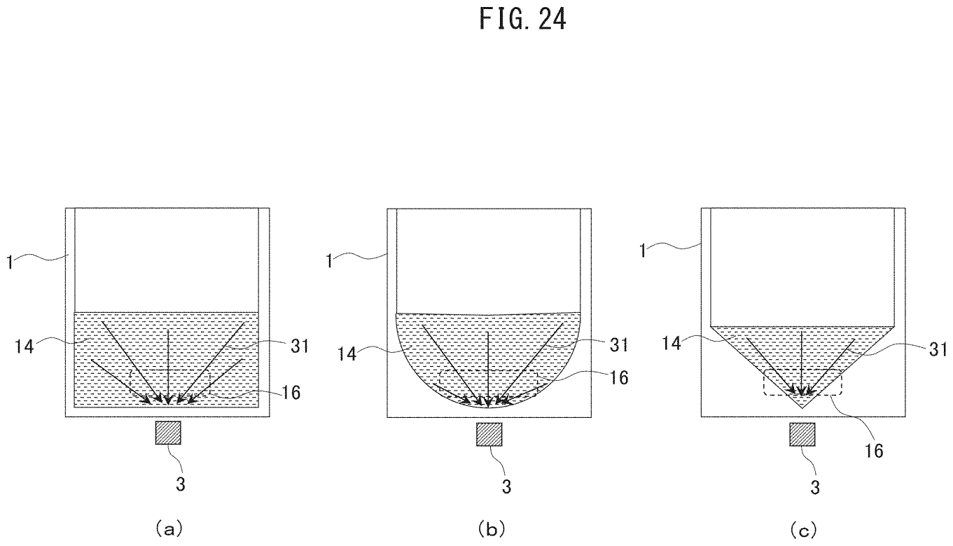

The following describes examples of the container used in the detection devices according to embodiments 1 to 3. The flat-bottomed container has mainly been described as an example of the container used in the above embodiments, but the container is not limited to this example. More specifically, the container 1 may have a curved bottom, as in FIG. 8, and have any shape without limitation. FIGS. 24(a) to (c) show side views of examples of the container used in the detection devices according to embodiments 1 to 3 of the present disclosure. FIGS. 24(a), 24(b), and 24(c) show side views of flat-bottomed, round-bottomed, and tapered containers, respectively.

The shapes shown in FIGS. 24(a) to (c) are merely examples, and the container is not limited thereto. More specifically, the shape is not limited to flat-bottomed, round-bottomed, nor tapered, and may be one halfway between them. Further, the ratio of particles passing through the detection region can be increased by setting the shapes of the taper and the magnet so that magnetic force may act along the taper.

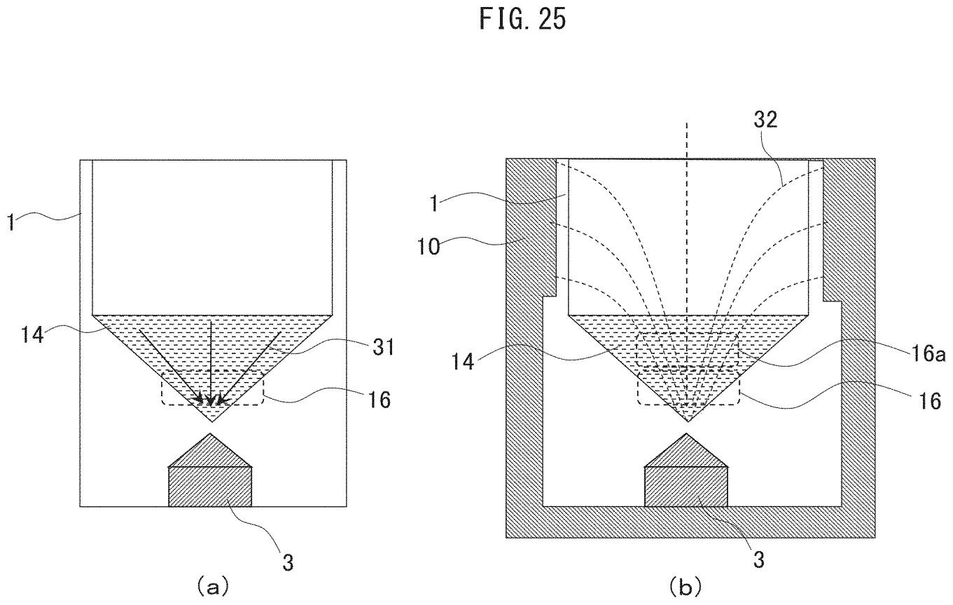

FIG. 25 shows an example of side views of the container and the magnetic field generating unit used in the detection devices according to embodiments 1 to 3 of the present disclosure. FIG. 25(a) shows an example in which they include a sharp-pointed magnetic field generating unit, and FIG. 25(b) shows an example in which the container includes a yoke in the case of FIG. 25(a).

As shown in FIG. 25(a), sharpening the tip of the container 1 enables the substance to be measured to be concentrated into a single point at the bottom of the container 1, allowing for improving the efficiency of concentration. Additionally, as shown in FIG. 25(b), providing a yoke 10 allows for increasing the magnetic field intensity of the magnetic field generating unit 3. Additionally, making the shape of the bottom of the container 1 agree with that of magnetic lines 32 of force enables magnetic paths to be concentrated in the detection region 16, increasing the efficiency of detection. FIG. 25(b) shows the example in which the detection region 16 includes part of the bottom of the container 1, but it is not limited to this example. As indicated by 16a, the detection region may be separated from the bottom of the container 1.

The following describes specific examples of the steps of detection, taking candida, E. coli, and CRP (C-reactive protein) as examples of the substance 11 to be measured.

### Example 1

An example in which candida is detected without using a fluorescent labeling substance will be described. The size of candida, which is a fungus, is approximately 5 to 10 [μm]. Candida is an indigenous fungus inhabiting, for example, the saliva, body surface, and digestive tract of humans. As shown in FIG. 1, 4 [μL] of sample solution containing candida, the substance 11 to be measured, and 4 [μL] of PBS solution, the magnetic labeling substance 12, to which Candida albicans antibody is bound are mixed in the container 1 as the solution 14 to make composite particles. Candida albicans antibody labeled with biotin may be bound to (mixed and reacted with) candida, the substance 11 to be measured, and then a magnetic labeling substance 12 labeled with avidin may be bound thereto.

Candida albicans antibody labeled with biotin is obtained by combining Anti-Candida albicans, Mouse (B341M)_IgG available from GeneTex Inc., with EZ-Link NHS-LC-Biotin available from Thermo Fisher Inc. As the magnetic labeling substance 12 labeled with avidin was used Dynabeads M-280 Streptavidinis available from Invitrogen Corp.

The mixed solution 14 reacts in the container 1 where convection occurs, forming composite particles 13 composed of candida, Candida albicans antibody, and the magnetic labeling substance 12. The flow generating unit 2 (e.g., convection, movement or rotation of the container, a flow cell, or gravity), which generates a flow in the first direction 21 in the solution 14 may be a means for generating the convection. For example, illumination light 51 from the illumination device 5 causes convection, generating a flow in the first direction 21.

When an external magnetic field is applied to the container 1, the magnetic labeling substance 12 exhibits characteristic motion. More specifically, the composite particles 13 including candida, the substance 11 to be measured, and the magnetic labeling substance 12 exhibit characteristic motion. The magnetic field generating unit 3 (e.g., a magnet, an electromagnet, or a magnetic film), which generates a magnetic field gradient in the detection region 16, may be used as a means for generating the external magnetic field.

It is irradiated by the illumination device 5 with spatial light (either transmitted light or epi-illumination light will do) as the illumination light 51, and detection light 41 reflected by the composite particles 13 is observed by the detection unit 4 at magnification of 50 to 1000. Then, the shapes and behavior of the composite particles 13, the magnetic labeling substance 12, and other substances can be seen. The composite particles 13 including candida can be distinguished by the shape specific to candida (yeast-like or mycelioid shape), the shape of the composite particles 13, and the characteristic motion in the second direction 31 different from the first direction 21 caused by the external magnetic field. Quantitative detection of candida, the substance 11 to be measured, could be achieved by obtaining two-dimensional images, using a means for optical detection (e.g., an image sensor) as the detection unit 4, and further analyzing the images.

The following describes the magnetic labeling substance used in the detection devices and methods according to the examples of the present disclosure. The magnetic labeling substance 12 has a structure of magnetic beads used for biomedical application, and as the magnetic substance, spinel ferrite is generally used. The size of the magnetic labeling substance 12 varies from nanometers to micrometers. A nano-sized substance has a larger surface area and diffuses wider in the solution on average by the Brownian movement, resulting in a high reactivity with the substance to be measured. However, since its particle size is small, magnetic force is weak. As the magnetic labeling substance 12, one having a size of 10 [nm] to 10 [μm] can be used.

### Example 2

An example in which E. coli is detected without using a fluorescent labeling substance will be described. E. coli, which is a bacterium, has a minor axis of 0.4 to 0.7 [μm] and a major axis of 2.0 to 4.0 [μm]. It is one of major species of bacteria existing in the environment. As shown in FIG. 1, 5 [μL] of sample solution containing E. coli, the substance 11 to be measured, and 5 [μL] of PBS solution of Dynabeads anti-E. coli 0157 available from Thermo Fisher Inc., as the magnetic labeling substance 12, are mixed in the container 1 as the solution 14.

The mixed solution 14 reacts in the container 1 where convection occurs, forming composite particles 13 composed of E. coli, an anti-E. coli antibody, and the magnetic labeling substance 12. The flow generating unit 2 (e.g., convection, movement or rotation of the container, a flow cell, or gravity), which generates a flow in the first direction 21 in the solution 14 may be a means for generating the convection. For example, illumination light 51 from the illumination device 5 causes convection, generating a flow in the first direction 21. After that, a magnetic field is applied by the magnetic field generating unit 3 to move the composite particles 13 in the second direction 31, and the composite particles 13 are detected with the illumination light 51, which is spatial light. These steps are similar to those in the case of candida described above, and thus description thereof is omitted.

### Example 3

An example in which candida is detected using a fluorescent labeling substance will be described. As shown in FIG. 13, 4 [μL] of sample solution containing candida, the substance 11 to be measured, 2 [μL] of fluorescent stain solution containing a fluorescent labeling substance 15, and 2 [μL] of PBS solution, the magnetic labeling substance 12, are mixed in the container 1 as the solution 14. As a fluorescent labeling reagent that is a fluorescent stain solution containing the fluorescent labeling substance 15, was used Fungiflora Y, which is a fluorescent stain solution for fungi, available from Trustmedical Co. Ltd.

Candida albicans antibody labeled with biotin is obtained by combining Anti-Candida albicans, Mouse (B341M)_IgG available from GeneTex Inc., with EZ-Link NHS-LC-Biotin available from Thermo Fisher Inc. As the magnetic labeling substance 12 labeled with avidin was used Dynabeads M-280 Streptavidinis available from Invitrogen Corp. Moreover, a method with a fluorescent labeling substance or a method of applying fluorescence resonance energy transfer may be used as a means for fluorescent labeling.

The antibody may be β1,3-glucan antibody or others that react specifically to a fungus, besides Candida albicans antibody.

The mixed solution 14 reacts in the container 1 where convection occurs, forming composite particles 13e composed of fluorescent candida, Candida albicans antibody, and the magnetic labeling substance 12. The flow generating unit 2 (e.g., convection, movement or rotation of the container, a flow cell, or gravity), which generates a flow in the first direction 21 in the solution 14 may be a means for generating the convection. For example, illumination light 51 from the illumination device 5 causes convection, generating a flow in the first direction 21.

When an external magnetic field is applied to the container 1, the magnetic labeling substance 12 exhibits characteristic motion. More specifically, the composite particles 13e including candida, which is the substance 11 to be measured, the magnetic labeling substance 12, and the fluorescent labeling substance 15 exhibit characteristic motion in the second direction 31 different from the first direction 21. The magnetic field generating unit 3 (e.g., a magnet, an electromagnet, or a magnetic film), which generates a magnetic field gradient in the detection region 16, may be used as a means for generating the external magnetic field.

It is irradiated by the illumination device 5 with spatial light having an excitation wavelength of the fluorescent labeling substance (either transmitted light or epi-illumination light will do) as the illumination light 51, and fluorescent detection light 41 reflected by the composite particles 13e is observed by the detection unit 4 at magnification of 50 to 1000. Then, the composite particles 13e including the fluorescent labeling substance 15 and an unreacted fluorescent labeling substance 15 can be observed as light spots. Additionally, the composite particles 13e including the magnetic labeling substance 12 can be distinguished by the characteristic motion in the second direction 31 different from the first direction 21 caused by the external magnetic field. Quantitative detection of candida, the substance 11 to be measured, could be achieved by obtaining two-dimensional images, using a means for optical detection (e.g., an image sensor) as the detection unit 4, and further analyzing the images. Combining a light source of a fluorescence wavelength with other wavelengths, such as white light, allows for obtaining information on the shapes of cells and the background together with information on fluorescent light and motion, which is effective in detecting a complex sample solution.

The following describes the fluorescent labeling substance used in the detection devices and methods according to the examples of the present disclosure. As the fluorescent labeling substance, one having a size of 10 [nm] to 10 [μm] can be used. It is expected that a fluorescent labeling substance 15 labeled with a fluorochrome, such as fluorescein (FITC), has high reactivity, because it has a smaller size than the magnetic labeling substance 12. For this reason, if the fluorescent labeling substance 15 and the magnetic labeling substance 12 are simultaneously added to the solution 14 to start a composite reaction, the fluorescent labeling substance 15 will react faster, which may reduce the magnetic labeling substance 12 that binds to the surface of the substance 11 to be measured.

To prevent this, it would be desirable to add the magnetic labeling substance 12 first to make it react, and then add the fluorescent labeling substance 15. In other words, it is expected that the smaller fluorescent labeling substance 15 can enter space between particles of the magnetic labeling substance 12 that is bound to the substance 11 to be measured. To larger particles, the magnetic labeling substance 12 acts as a three-dimensional barrier. In other words, imbalance of reactions can be prevented by changing the order of reactions according to the size of particles.

### Example 4

An example in which E. coli is detected using a fluorescent labeling substance will be described. As shown in FIG. 13, a sample solution containing E. coli, which is the substance 11 to be measured, an anti-E. coli antibody labeled with the fluorescent labeling substance 15, and Dynabeads anti-E. coli O157 available from Thermo Fisher Inc., which is an anti-E. coli antibody, magnetically labeled with the magnetic labeling substance 12 are mixed in the container 1 as the solution 14. The fluorescent anti-E. coli antibody is obtained by combining Anti-E. coli antibody (Biotin) available from Abcam plc., with 1.0 [μm] of Streptavidin Microspheres available from Polysciences Inc.

The mixed solution 14 reacts in the container 1 where convection occurs, forming composite particles 13e composed of the fluorescent labeling substance 15, E. coli, and the magnetic labeling substance 12. The flow generating unit 2 (e.g., convection, movement or rotation of the container, a flow cell, or gravity), which generates a flow in the first direction 21 in the solution 14 may be a means for generating the convection. For example, illumination light 51 from the illumination device 5 causes convection, generating a flow in the first direction 21. After that, a magnetic field is applied by the magnetic field generating unit 3 to move the composite particles 13e in the second direction 31, particles to which the fluorescent labeling substance 15 is bound are detected with the illumination light 51, which is spatial light, and the composite particles 13e are detected, based on motion of the detected particles. These steps are similar to those in the case of candida described above, and thus description thereof is omitted.

### Example 5

An example in which CRP is detected using a fluorescent labeling substance will be described. As shown in FIG. 13, an anti-CRP antibody magnetically labeled with the magnetic labeling substance 12 and an anti-CRP antibody labeled with the fluorescent labeling substance 15 are added to a sample solution containing CRP, the substance 11 to be measured, as the solution 14, to form composite particles 13e. The composites can be formed by using an anti-CRP antibody labeled with a fluorescent substance, FITC, as the fluorescent anti-CRP antibody or using an anti-CRP antibody labeled with biotin and fluorescence beads labeled with avidin that are reacted beforehand as the fluorescent CRP antibody. Besides these examples, various fluorochromes are available, such as FITC, PE, rhodamine, Cy pigment, and AlexaR, and one whose excitation wavelength and fluorescence wavelength differ can also be used. After that, a magnetic field is applied by the magnetic field generating unit 3 to move the composite particles 13e in the second direction 31, particles to which the fluorescent labeling substance 15 is bound are detected with the illumination light 51, which is spatial light, and the composite particles 13e are detected, based on motion of the detected particles. These steps are similar to those in the case of candida, and thus description thereof is omitted.

The above detection devices and methods according to the examples of the present disclosure enable detection of micron-sized bacteria, fungi, and other substances in a solution.

1. A detection device comprising:
a container that retains a solution containing a substance to be measured and a magnetic labeling substance that binds specifically to the substance to be measured;
a flow generating unit that generates a flow in a first direction at least in the solution;
a magnetic field generating unit that generates a magnetic field gradient in the solution; and
a detection unit that detects composite particles, based on motion of particles in a predetermined region in the solution, the composite particles including the substance to be measured and the magnetic labeling substance bound together.

2. The detection device according to claim 1, wherein the predetermined region in the solution is separated from an inner wall surface of the container.

3. The detection device according to claim 1, wherein the flow generating unit is a light source that radiates spatial light into the container.

4. The detection device according to claim 1, wherein
the solution contains another substance that is not the substance to be measured nor the magnetic labeling substance, and
the detection unit detects the composite particles, based on motion of the composite particles and the other substance in the predetermined region in the solution.

5. The detection device according to claim 1, wherein the magnetic field generating unit moves the composite particles in a second direction different from the first direction.

6. The detection device according to claim 1, wherein the magnetic field generating unit moves the composite particles in a second direction identical to the first direction.

7. The detection device according to claim 4, wherein the detection unit detects the composite particles, based on directions of motion of the composite particles and the other sub stance.

8. The detection device according to claim 4, wherein the detection unit detects the composite particles, based on speeds of motion of the composite particles and the other substance.

9. The detection device according to claim 1, wherein the flow generating unit heats the solution to cause convection therein to generate the flow in the first direction at least in part of the solution.

10. The detection device according to claim 1, wherein the flow generating unit rotates the container to generate the flow in the first direction at least in part of the solution.

11. The detection device according to claim 1, wherein the flow generating unit stirs the solution to generate the flow in the first direction at least in part of the solution.

12. The detection device according to claim 1, wherein
the composite particles further include a fluorescent labeling substance, and
the detection unit detects the fluorescent labeling substance to detect particles to which the fluorescent labeling substance is bound, and
the detection unit detects the composite particles, based on motion of the detected particles.

13. A detection method comprising the steps of:
retaining in a container a solution containing a substance to be measured and a magnetic labeling substance that binds specifically to the substance to be measured;
generating a flow in a first direction at least in the solution;
generating a magnetic field gradient in the solution; and
detecting composite particles, based on motion of particles in a predetermined region in the solution, the composite particles including the substance to be measured and the magnetic labeling substance bound together.
